(19) United States
(12) Patent Application Publication
(10) Pub. No.: US 2025/0154591 A1
(43) Pub. Date: May 15, 2025

(54) Method for detecting and subtyping inflammatory intestinal diseases

(71) Applicant: Åbo Akademi, Åbo (FI)

(72) Inventors: Lauri Polari, Åbo (FI); Diana Toivola, Åbo (FI); Terhi Helenius, Åbo (FI)

(21) Appl. No.: 18/838,621

(22) PCT Filed: Feb. 17, 2023
(86) PCT No.: PCT/FI2023/050098

§ 371 Date: Aug. 15, 2024

(30) Foreign Application Priority Data
Feb. 18, 2022 (FI) 20225151

(51) Int. Cl. C12Q 1/6883; C12Q 1/6851; G01N 33/68

(52) U.S. Cl. CPC C12Q 1/6883; C12Q 1/6851; G01N 33/6893; C12Q 2600/112; G01N 2333/4742; G01N 2800/065

(57) ABSTRACT

The present invention is related to a method for determining or confirming chronic inflammatory intestinal disease or a risk thereof in a subject. The method comprises detecting the presence of keratin 7 (K7) mRNA or protein in a biological sample obtained from a subject.

### FIELD

The present invention relates generally to diagnostics methods and particularly to methods for detecting intestinal inflammation and identifying its subtype disease.

### BACKGROUND

The prevalence of chronic inflammatory bowel disease (IBD) has been increasing globally, affecting over 0.3% of the population in western countries. Both ulcerative colitis (UC) and Crohn's disease (CD) induce severe symptoms and may lead to further complications and increase the risk of large intestine tumors. Rapid and accurate diagnosis methods to estimate the severity of the disease are constantly required to select the most efficient treatments and improve the outcome as well as to predict the course of illness. As IBD incidence rate is growing most rapidly in developing countries, especially cost-efficient methods are highly needed.

Calprotectin concentration in stool has been used as a “golden standard” marker for IBD associated inflammation. However, calprotectin result does not distinguish between the subtypes of IBD, or evaluate the severity or prediction of the disease (State et al. 2021, Freeman et al. 2019). The colonic epithelial barrier is often severely compromised in IBD immune cell flux (De Souza and Fiocchi, 2016), witnessed by erosion, edema and later regeneration and hyperplasia to reinstate cellular integrity and tissue homeostasis. Currently, there are no valid colon epithelial cell produced compounds utilized to indicate the local inflammatory responses, similar to, e.g., liver epithelia produced CRP.

Among the integrity maintaining epithelial cell components, intermediate filament keratins are key cytoskeletal proteins. There are 56 different keratins in humans and their expression profiles are tissue specific. Keratins K8, K18, K19 and K20 are dominant in human colon and K23 can be found at minor level. Typically the most tissue specific keratin patterns are stable in general, for instance in tumorigenesis. Keratin staining is utilized in cancer diagnostics to identify the tissue of origin of metastasis. To our knowledge keratin profiles have not been use in the diagnostics of inflammatory intestinal diseases.

WO200259367 discloses a diagnostic microarray for inflammatory bowel disease, Crohn's disease and ulcerative colitis. In this method, RNA from mononuclear blood cells is analyzed to determine over- and under-expression of gene sequences.

### SUMMARY OF THE INVENTION

The aim of this invention is to overcome the limitations in the diagnostic tools for inflammatory intestinal diseases. The invention is based on a novel finding that keratin levels are altered in the colon of patients with inflammatory bowel disease. Particularly, the level of keratin 7 is increased in such patients. The present disclosure provides a method where changes in epithelial gene expression is used to diagnose and classify inflammatory intestinal diseases and its subtypes and conditions. The present invention may also be utilized in predicting disease progression and designing personal medication for IBD patients.

According to the first aspect of the present invention, a method for determining or confirming chronic inflammatory intestinal disease or a risk thereof in a subject is provided. The method comprises detecting the presence of keratin 7 (K7) mRNA or protein in a biological sample obtained from a subject.

According to the second aspect of the present invention, use of a K7-specific binder in the ex vivo diagnosis of chronic inflammatory intestinal disease or subtypes thereof for the detection of the presence of the K7 protein or mRNA in a gastrointestinal tract sample or a stool sample is provided.

The invention is defined by the features of the independent claims. Some specific embodiments are defined in the dependent claims.

### BRIEF DESCRIPTION OF THE DRAWINGS

FIG. 1. K7 is upregulated in UC and CD. (A) The ratio of K7 expressing epithelial cells is increased both in ulcerative colitis (UC) and Crohn's disease (CD), a dot represents a single patient from the cohort A. Single cell intensities from the same samples shown in a histogram (B) and as min-max floating bars with mean (C) show that both minimum, maximum and mean relative K7 intensities are increase in CD and UC. Maximum K7 values are also increased in collagenous colitis (CC). (D) Similar result is seen when K7+ positive cells are split in three grades according to the cellular K7 intensity. Values refer to the percentage of epithelial in each grade, calculated per patient. Box extends from 25th to 75th percentile, line inside shows median value and whiskers min and max values. (E) Typical K7 expression rates and patterns shown in representative K7 IHC figures. Respective HE stainings show associated disease pathologies. Scale bar=50 μm. Statistical significance is based on Dunn's multiple comparison test. *P<0.05; **P<0.01; ***P<0.001.

FIG. 2. Keratin-7 mRNA is expressed in inflamed colon but not in healthy colon. (A) According to the tissue-specific mRNA expression in bulk RNAseq, dataset published by Su et al. 2004, Keratin-7 mRNA (probe 209016_s_at) is not found at detectable levels in tissues of intestinal origin (arrows). (B and C) Keratin-7 is significantly upregulated in IBD patient's colon tissue. Data is downloaded from publicly available transciptomes ((B), Arijs et al. 2009, E-GEOD-14580; (C) Galamb et al. 2008, E-GEOD-4183) which include samples from both IBD patients and healthy controls. A dot represents a single individual. Box extends from 25th to 75th percentile, line inside shows median value and whiskers min and max values. Statistical significance (B) was measured by Mann-Whitney test. *P<0.05; ***P<0.001.

FIG. 3. Correlation of clinical characteristics with K7 expression level. (A) Sex of the patient had no effect or correlation on K7 expression. (B) Mean crypt length correlated with K7 rate in the whole cohort. (C, D) When groups with and without significant hyperplasia were investigated separately, the individual K7 did not correlate with crypt length. Fecal calprotectin concentration prior surgery had no correlation to K7 in patients with CD (E) or UC (F). K7 upregulation was the most pronounced in drug resistant UC patients (G). Correlations were assessed using linear regression analysis. The significance between the groups was measured using Dunn's multiple comparison test. *P<0.05.

FIG. 4. Keratin 8 levels of patients by histological staining of colon and digital pathology analysis.

FIG. 5. Keratin detection at mRNA level in mouse stool after Keratin 8 deletion. Mouse K8 was downregulated in the floxed K8/Cre-villinERt2 transgenic mice and treatment of tamoxifen. The y-axis shows keratin fold change over time compared to day zero (normal keratin levels).

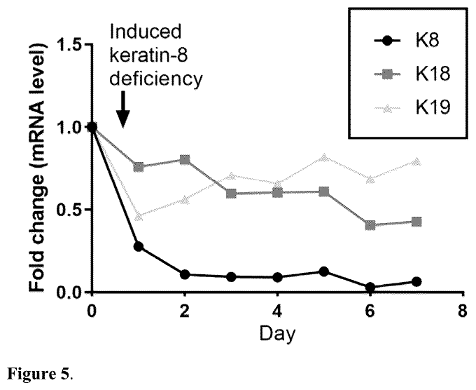

### EMBODIMENTS

As used herein, the term “antibody” encompasses naturally occurring and engineered antibodies, as well as full length antibodies, functional fragments, or analogs thereof that are capable of binding e.g., the target immune checkpoint or epitope (e.g. retaining the antigen-binding portion). The antibody may be from any origin including, without limitation, human, humanized, animal or chimeric, and may be of any isotype with a preference for an IgG1 or IgG4 isotype, and further may be glycosylated or non-glycosylated. The term antibody also includes bispecific or multispecific antibodies so long as the antibody(s) exhibit the binding specificity herein described.

The term “binder” within the context of the present disclosure may be understood as referring to polypeptides and other molecules, such as antibodies and aptamers or fragments thereof, having a potential capability of specifically binding other compounds and/or structures, in particular epitopes, more in particular peptidic epitopes in other proteins such as keratin 7. The binder may also be an oligonucleotide primer or probe specifically binding to K7 mRNA or cDNA derived from said K7 mRNA.

As used herein, the term “fragment” includes native peptides (either degradation products, synthetically synthesized peptides or recombinant peptides) and modified peptides, which may have, for example, modifications rendering the peptides more stable or less immunogenic. Such modifications include, but are not limited to, cyclization, N-terminus modification, C-terminus modification, peptide bond modification, backbone modification and residue modification. The fragment may also comprise further elongations, deletions, substitutions or insertions.

As used herein, the term “polypeptide” refers herein to any chain of amino acid residues, regardless of its length or post-translational modification (e.g., glycosylation or phosphorylation).

As used herein, the terms “subject,” “individual,” “host,” and “patient,” are used interchangeably herein to refer to an animal being treated with one or more exemplary compounds as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, including a human, that is suspected of having, has been diagnosed as having, or is at risk of developing a disease that can be ameliorated, treated or prevented by administration of one or more compounds known in the art to treat IBD. Preferably, said subject or patient is not known or suspected to suffer from cancer.

As used here in “keratin 7” and “K7” means keratin type II cytoskeletal 7 protein. Keratin 7 is also known by the names cytokeratin 7 and sarcolectin. Keratin 7 is encoded by the KRT7 gene. The amino acid sequence of human keratin 7 is found in Genbank under accession number NP_005547.3 and mRNA sequence under accession number NM_005556.4.

As used herein “keratin 8” and “K8” means keratin type II cytoskeletal 8 also known as cytokeratin-8. The amino acid sequence of human keratin 8 is found in Genbank under accession number NP_001243211.1 and mRNA sequence under accession number NM_001256282.2.

Inflammatory bowel diseases have been increasing globally. Rapid and accurate diagnosis methods are needed to estimate the severity of the disease so that the most efficient treatment can be selected. The present invention is directed to an ex vivo method for determining or confirming chronic inflammatory intestinal disease or a risk thereof in a subject. The method is based on detecting the presence of keratin 7 (K7) mRNA or protein in a biological sample obtained from a subject.

In some embodiments, the sample is selected from the group consisting of a stool sample or a gastrointestinal tract sample. Gastrointestinal tract sample may be, for example, a biopsy or a surgical removal. Preferably, the said gastrointestinal tract sample is a biopsy, more preferably a colon or small intestinal tissue biopsy.

The term “inflammatory intestinal disease” as used herein refers to chronic inflammation occurring in the intestines, and in a broad sense, may include all inflammatory diseases occurring in the intestines, such as infectious enteritis and ischemic bowel disease such as bacterial, viral, amoebic, or tuberculous enteritis, and the like; radiation enteritis;

and the like. Examples of inflammatory intestinal disease include but are not limited to irritable bowel syndrome, inflammatory enteritis, microscopic colitis, such as collagenous colitis (CC) and lymphocytic colitis (LC), and inflammatory bowel disease and its subtypes.

In the present specification, the term “inflammatory bowel disease” is used to mean a disease of unknown cause, wherein inflammation occurs in cells and affects the surface layer of the alimentary canal mucosa of the large intestine, small intestine, etc., and part of the mucosa is thereby lost, and as a result, ulcers or erosions are developed. Inflammatory bowel disease may be chronic. Specific examples of inflammatory bowel disease may include ulcerative colitis (UC) and Crohn's disease (CD). Typical examples of the ulcerative colitis may include intractable ulcerative colitis, fulminant ulcerative colitis and drug-resistant ulcerative colitis.

In some embodiments, the said inflammatory intestinal disease is an inflammatory bowel disease (IBD) or its subtype. Preferably, the IBD is Crohn's disease or ulcerative colitis, more preferably a drug-resistant ulcerative colitis. The presence of keratin 7 (K7) protein or mRNA in the sample confirms that the sample is associated with Crohn's disease or ulcerative colitis.

In some embodiments, the method distinguishes inflammatory bowel disease subtypes e.g. ulcerative Colitis and Crohn's disease from collagenous colitis, microscopic colitis and from irritable bowel syndrome. The presence of K7 protein and mRNA in the sample confirms that the sample is associated with inflammatory bowel disease subtypes. In other embodiments, the method comprises a step of differentiating microscopic colitis, preferably collagenous colitis or lymphocytic colitis, from inflammatory bowel disease, wherein the presence of K7 protein in the sample confirms that the sample is associated with inflammatory bowel disease.

In some embodiments, the presence of K7 protein and mRNA in the biological sample is determined by immunohistochemistry or in situ hybridization, respectively Preferably, the presence of K7 is determined by contacting said sample with a primary antibody specific to K7 and then the sample is visualized by further contacting said sample with a labelled secondary antibody binding to the primary antibody and a label-specific reagent. More preferably, the presence of the combination of the primary and secondary antibody is measured using an enzyme-linked immunosorbent assay (ELISA).

In some embodiments, the said ELISA is qualitative. In other embodiments, the said ELISA is quantitative. The method may comprise a step of diluting a stool sample in order to improve optical properties of the sample. In some embodiments, the sample is contacted with immobilized antibodies specific to the K7 protein to create a treated sample. Preferably, the treated sample is further contacted with enzyme-linked antibodies to create a readable sample. Most preferably, the optical density of said readable sample is determined at a suitable wavelength. In some embodiments, the method further comprises a step of generating a purified K7 protein standard curve. Preferably, optical density of the readable sample is compared to the standard curve to determine the concentration of the K7 protein in the sample, for example in a stool sample.

In other embodiments, the presence or expression of K7 is determined by preparing an RNA sample from the biological sample and detecting the presence and optionally the level of the K7 mRNA in said RNA sample. The presence of the K7 mRNA indicates that said sample is associated with inflammatory bowel disease.

In some embodiments, the amount of the K7 protein or K7 mRNA that is detected in the biological sample is compared to the amount of the K7 protein or K7 mRNA detected in corresponding samples taken from healthy population. Higher amount of the K7 protein or K7 mRNA detected in the biological sample than in the samples of healthy population confirms that the sample is associated with inflammatory bowel disease. In some embodiments, the amount of the K7 protein or K7 mRNA that is detected in the biological sample is compared with a cut-off value provided by corresponding assays performed to a number of subjects from healthy population. A value above the cut-off is an indication that the subject has IBD or a risk for developing IBD.

The presence of K7 in the patient sample may be detected with any method suitable of protein detection. In some embodiments, the presence of keratin 7 (K7) protein in the biological sample is determined by flow cytometry, mass cytometry, nuclear magnetic resonance (NMR), lateral flow assay (see e.g. WO2019215199) or by any immunofluorescence method.

In other embodiments, the method comprises an additional step, where the detection of K7 protein is combined with detection of a further biomarker in the samples. The further biomarker is selected from the group comprising keratin 8 (K8) protein, keratin 16 (K16) protein, keratin 17 (K17) protein, keratin 18 (K18) protein, keratin 19 (K19) protein, keratin 20 (K20) protein, keratin 23 (K23) protein, keratin 24 (K24) protein, keratin 80 (K80) protein and calprotectin. More preferably, said further biomarker is K8.

The present invention further relates to the use of a K7-specific antibody for the detection of the presence of the K7 protein. In some embodiments, K7 is detected from a gastrointestinal tract sample or a stool sample with a K7-specific antibody. Preferably, the said gastrointestinal tract sample is a colon tissue biopsy or a small intestinal tissue biopsy. In an embodiment said gastrointestinal tract sample is not a cancer sample or metastasis sample.

In other embodiments, the present disclosure is directed to a use of a K7-specific binder for the detection of the presence of the K7 protein in a stool sample or a gastrointestinal tract sample such as a biopsy, said gastrointestinal tract sample not being a sample or biopsy of cancer or cancer metastasis, wherein said binder is preferably an antibody or aptamer. In a preferred embodiment, said gastrointestinal tract sample is a colon tissue biopsy or a small intestinal tissue biopsy. A further embodiment of the present disclosure is directed to a binder specific to K7 protein or K7 mRNA for use in a method of diagnosis of chronic inflammatory intestinal disease or subtypes thereof, wherein said binder is preferably an antibody or aptamer for said protein or an oligonucleotide probe for said mRNA.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting. Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### EXPERIMENTAL SECTION

### Material and Methods

### Patient Material.

Patient cohort A samples were obtained from the Auria Biobank (Turku, Finland). Transport, handling and storage of the paraffin-embedded patient tissue samples were carried out in a standard way according to the biobank guidelines. The medical history of every patient, relevant to IBD, was filed and information was stored in encoded format and thus kept anonymous. The research project was authorized by the Auria Biobank's Scientific Steering Committee (AB17-6901) and Hospital District of Southwest Finland (T05/032/19). Bulk RNAseq data was accessed using biogps.org gene annotation portal.

### Immunohistochemistry and Pathological Evaluation.

Prior IHC staining, pathologist inspected biobank template H&E stained slides of every biopsy to confirm the original diagnosis, the adequate presence of intestinal epithelial cells and orientation with the focus to view several full top to bottom crypts. The control tissues were collected from patients with the exclusion criteria of IBD and neoplastic intestinal diseases. UC and CD sample were harvested during colectomy or ileum resection. CC, LC and control samples were biopsies harvested during ileocolonoscopy. The clinical characteristics and medications of cohort A is presented in Table 1.

TABLE 1
Clinical characteristics and medication of patients. The number in “Number of drug receivers”
columns indicate the number of patients receiving this medication within 1 month prior to tissue harvest.

| | | | Age at | Years from | | | Number of drug receivers | | | | | | |
|---|---|---|---|---|---|---|---|---|---|---|---|---|---|
| | | Sex | harvest | onset to harvest | BMI | | | Cortico- | TNFα | Azathi- | Sulfa- | Metho- | |
| Group | Number | F/M | (median) | (median) | (median) | | Mesalazine | steroids | blockers | oprine | salazine | trexate | Other |
| Ctrl | 12 | 6/6 | 34-80 (57) | — | 18.3-28.7 | (27.6)* | 1 | 0 | 0 | 0 | 0 | 0 | |
| UC | 15 | 6/9 | 15-85 (47) | 0-29 (6) | 19.1-29.8 | (24.9) | 12 | 14 | 5 | 6 | 2 | 0 | *** |
| CD | 11 | 6/5 | 17-78 (35) | 0-22 (6) | 17.2-38.1 | (22.6) | 5 | 5 | 3 | 3 | 1 | 2 | |
| CC | 8 | 6/2 | 40-85 (78) | 0-9 (0) | 23.4-23.5 | (23.5)** | 2 | 3 | 0 | 0 | 1 | 0 | **** |
| LC | 10 | 5/5 | 63-88 (78) | 0-4 (0) | 19.3-28.9 | (25.3) | 0 | 0 | 0 | 0 | 0 | 0 | |

*only 4 values available;
**only 2 values available;
*** single user of Allupurinol, Ursodiol and anabolic steroids;
**** single user of Leflunomide and Hydroxychloroquine

The tissue samples were fixed in 4% phosphate-buffered formaldehyde and embedded in paraffin according to standard procedures. K7 and K8 IHC stainings were carried out from 5 μm rehydrated sections with antibody to K7 (clone SP52, Roche Diagnostics) and to K8 (clone CAM 5.2, Becton Dickinson). The protein visualization was carried out using anti-mouse secondary antibody and 3,3′-Diaminobenzidine (DAB) as a chromogen and hematoxylin counter-stain. Calprotectin immunoassays were carried out from stool samples in local hospital laboratories.
For grading of the severity of IBD, histology was used as the main reference standard. The inflammation activity in samples were graded into four classes: no activity (remission), mild activity (ad cryptitis), moderate activity (crypt abscesses) and severe (erosion/ulcers) according to ECCO guidelines (Magro et al., 2013).

### Digital Image Analysis.

The slides were scanned (Pannoramic 1000, 3D HISTECH, Budapest, Hungary) and pathological changes were evaluated by at least two researchers. The quantification of K7 positive cells and the mean intensity of cellular K7 were measured using QuPath 0.2.3 bioimage analysis application (Bankhead et al., 2017). The epithelial cell layer region of interest (ROI) to be quantified was selected manually excluding immune, mesenchymal, endothelial and muscle cells from at least two distinct areas per samples, both including lumen and crypts, full crypts being prioritized when available. This epithelial ROI was chosen to contain over 2000 epithelial cells per sample, identified using the QuPath cell detection tool. Cellular K7 expression is based on mean intensity of cellular DAB staining. In addition, every cell in ROI areas was ranked according to the K7 intensity and given a value from 0-3, where 0=no K7 present, 1=low K7, 2=medium and 3=high K7 expression rate, using the QuPath positive cell detection (DAB cell mean OD) tool. The lowest threshold was based on barely visible cytoplasmic DAB staining and the upper value on intensity surpassing respective nuclear hematoxylin staining. The cells ranked 1-3 for K7 are referred to here as K7 positive. Crypt length measurements is an average of at least four full top-to-bottom crypts/patient, measured from digital HE stained samples. The crypt length was measured from digitally scanned HE samples, consisting average of at least four full crypts.

### Statistical Analysis.

The difference between more than two groups was measured using Kruskal-Wallis test, followed by Dunn's multiple comparison. The difference between two factors was measured using Mann Whitney test. The linear correlation of two factors was studied using linear regression analysis.

### Mice Experiments.

Stool samples were collected from K8^{flox/flox}, Villin-CreERt2 mice, which possess tamoxifen-inducible keratin-8 deficiency. Stools samples were collected daily after tamoxifen induction and stored in −80° C. Total RNA was extracted from stool samples using NucleoSpin RNA Stool kit (Macherey Nagel, Germany). RNA was quantified by NanoDrop assay and reverse transcribed into cDNA using cDNA synthesis kit (Promega, Madison, WI). Genes of interest were amplified using QuantStudio™ 3 real-time PCR system (Applied Biosystems™, CA, USA) with designed primers (Stenvall et al. 2022) and SensiFAST SYBR Hi-ROX Kit (Meridian Bioscience, Cincinnati, OH, USA).

### Results

### K7 is De Novo Expressed in the Colonic Epithelium in UC and CD Patients.

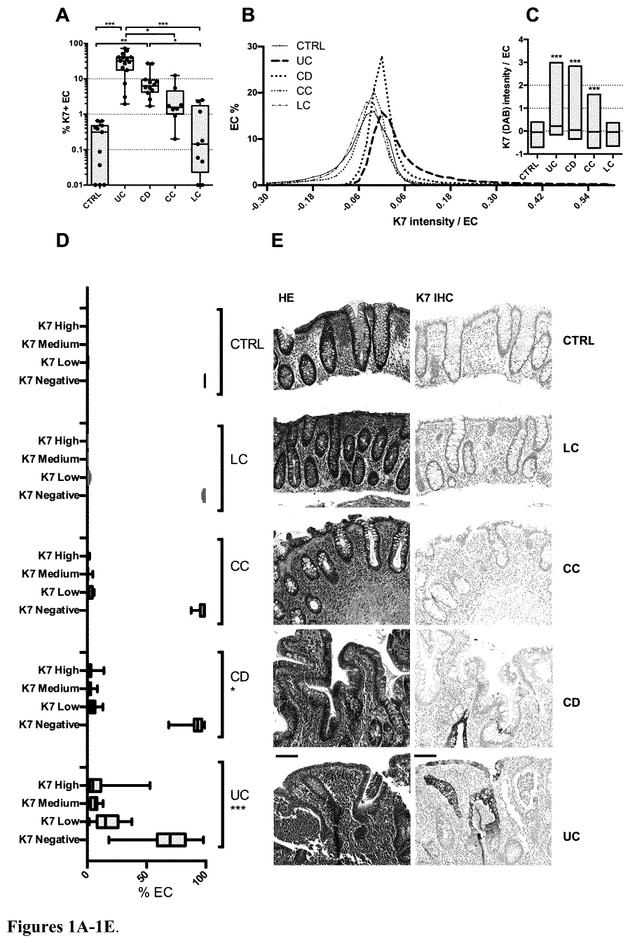

To analyze if K7 is expressed in the colon of IBD patients, histological sections were stained for K7 and levels analyzed using quantitative digital analysis. K7 protein expression in the colon was upregulated in UC and CD, compared to controls and the microscopic colitis sub-disease collagenous (CC) and lymphocytic colitis (LC) (FIG. 1A), while the levels of K7 in CC and LC were not significantly different from controls (FIG. 1A). Analysis of single cell K7 levels revealed that both the number of K7 positive cells as well as the mean cellular concentration of K7 were increased in UC and CD (FIG. 1B-D). Similar result is seen when K7+ positive cells are split in three grades according to the cellular K7 intensity. K7 positive cell number increased slightly in CC patients, but K7 expression levels in LC patients were similar to healthy controls, i.e. less than 1% of the epithelial cells being K7 positive (FIG. 1A, D). Also, the number of cells with high K7 expression were only found in UC and CD samples (FIG. 1C, E), suggesting that cellular K7 concentration is increased respectively to K7 positive cell density. Representative HE and K7 stainings are shown in FIG. 1E.

K7 Upregulation in IBD is Detectable at mRNA Level.

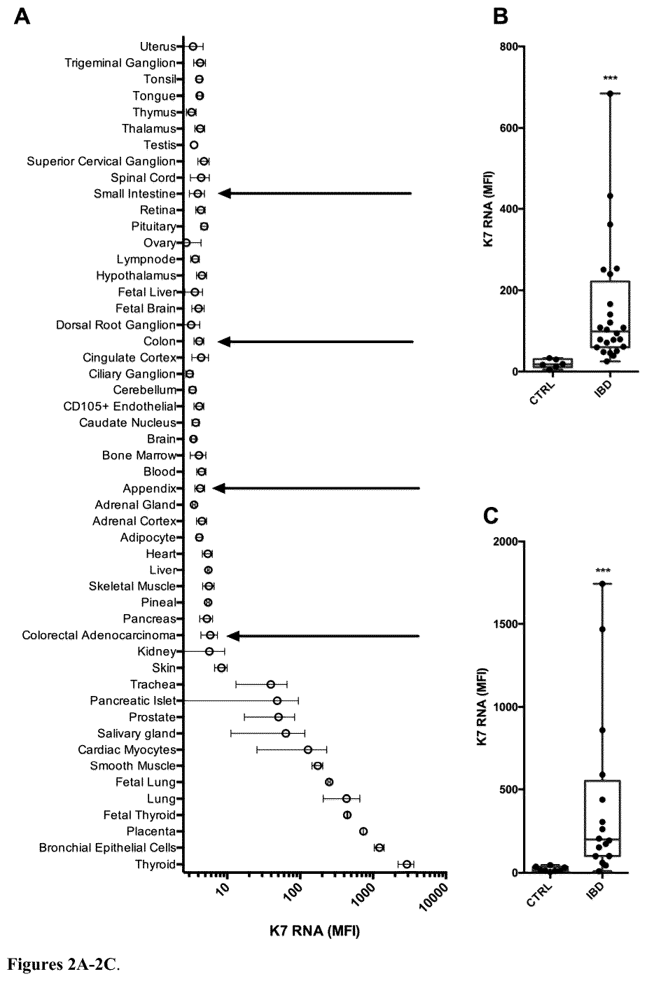

To study the K7 mRNA expression, we analyzed the publicly available human gene atlas data (Su et al., 2004), which suggests that K7 is not significantly expressed in healthy intestine at mRNA level (FIG. 2A). To find out whether K7 expression in IBD was regulated at transcription or post translationally, we quantified the K7 mRNA data from bulk RNAseq data E-GEOD-14580 (Arijs et al., 2009) and E-GEOD-4183 (Galamb et al., 2008) in ArrayExpress repository. In both cases, K7 mRNA levels are significantly increased in the colon of IBD patients (FIG. 2B, 2C) compared to controls. As K7 was increased at both protein and RNA level in IBD, it suggests that inflammation associated colon K7 increase is regulated at least party on transcriptional level.

### Colonic K7 Level Increase is Independent of Clinical Characteristics but Increased in Hyperplasia.

Next, we wanted to study whether the K7 increase is linked with clinical characteristics of patients. Sex, age, BMI and time from disease onset had no correlation with the relative K7+ cell density in IBD. Calprotectin level or the duration of the disease from onset to tissue harvest were neither definitive.

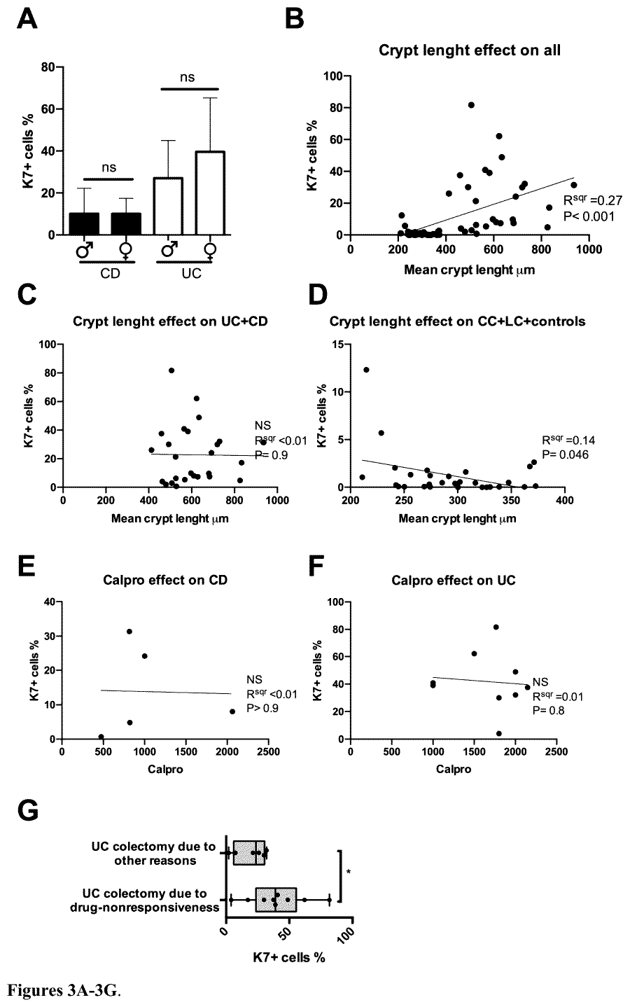

K7 expression was higher in general in CD and UC, which include significantly increased crypt length, compared to LC and CC (FIG. 3). Nevertheless, the correlation was diminished when only samples with or without hyperplasia were compared internally to the number of K7 positive cells (FIG. 3). Interestingly, the K7+ cell location in crypts did not seem to have a clear dimensional pattern. While K7 positive cells often occur in clusters next to each other, we also found several sporadic cells with K7 positivity.
K7 Expression Increase is Associated with Drug-Resistant Colitis.

Patients whose colectomy was carried out due to drug resistance, possessed higher number of K7+ cell to those whose colectomy was due to other reasons including cancer, major dysplasia and severe infection (FIG. 3). There was stool calprotectin difference between UC and CD patients, while fecal calprotectin level did not correlate with epithelial K7 expression neither (FIG. 3).

### Keratin 8 Levels Vary in Inflammatory Intestinal Diseases.

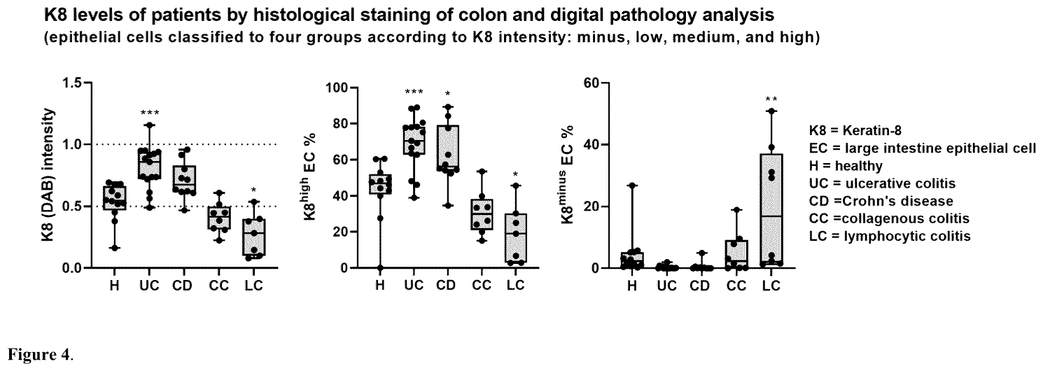

To analyze whether colonic K8 is changed in IBD and microscopic colitis patients, histological sections were stained for K8 and levels analyzed using quantitative digital analysis similarly to K7 analysis. K8 protein expression in the colon was upregulated in UC and CD, compared to controls and the microscopic colitis sub-disease collagenous (CC) and lymphocytic colitis (LC) (FIG. 4), witnessed by increased mean cellular K8 intensity as well as higher the number of high K8 expression level cells.

Keratin can be Detected at mRNA Level from Stool.

Keratin RNA is quantitatively measurable from stool samples. Mouse study showing that keratins (K) can be detected from stool samples even at mRNA level, fold change to day zero (normal keratin levels). Mouse intestinal K8 was conditionally downregulated using floxed K8 and villin Cre-ert2 gene construct which was activated in vivo with tamoxifen. The change in K8 expression can be seen from stool samples immediately, while K18 and K19 mRNA remained close to basal level. The downregulation of K8 found in stool after conditional knockdown was found similarly what has been shown earlier in tissue level in the colon (Stenvall et al. 2022). This suggests that keratins can be quantified from stool samples thus enabling non-invasive analysis.

### CITATION LIST

### Patent Literature

WO200259367
WO2019215199

### Non Patent Literature

Arijs, I., Li, K., Toedter, G., Quintens, R., Van Lommel, L., Van Steen, K., Leemans, P., De Hertogh, G., Lemaire, K., Ferrante, M., et al. (2009). Mucosal gene signatures to predict response to infliximab in patients with ulcerative colitis. Gut 58, 1612-1619.
De Souza, H. S. P., and Fiocchi, C. (2016). Immunopathogenesis of IBD: Current state of the art. Nat. Rev. Gastroenterol. Hepatol. 13, 13-27.
Freeman, K., Willis, B. H., Fraser, H., Taylor-Phillips, S., and Clarke, A. (2019). Faecal calprotectin to detect inflammatory bowel disease: A systematic review and exploratory meta-analysis of test accuracy. BMJ Open 9, 1-11.
Galamb, O., Györffy, B., Sipos, F., Spisák, S., Németh, A. M., Miheller, P., Tulassay, Z., Dinya, E., and Molnár, B. (2008). Inflammation, adenoma and cancer: Objective classification of colon biopsy specimens with gene expression signature. Dis. Markers 25, 1-16.
Magro, F., Langner, C., Driessen, A., Ensari, A., Geboes, K., Mantzaris, G. J., Villanacci, V., Becheanu, G., Nunes, P. B., Cathomas, G., et al. (2013). European consensus on the histopathology of inflammatory bowel disease. J. Crohn's Colitis 7, 827-851.
Moll, R., Krepler, R., and Franke, W. W. (1982). Complex Cytokeratin Polypeptide Patterns Observed in Certain Human Carcinomas. Differentiation 23, 256-269.
State, M., Negreanu, L., Voiosu, T., Voiosu, A., Balanescu, P., and Mateescu, R. B. (2021). Surrogate markers of mucosal healing in inflammatory bowel disease: A systematic review. World J. Gastroenterol. 27, 1828-1840.

Stenvall, C., Tayya, M., Grönroos, T., Ilomäki, M., Viiri, K., Ridge, K. M., Polari, L., and Toivola, D. M. (2022). Targeted deletion of keratin 8 in intestinal epithelial cells disrupts tissue integrity and predisposes to tumorigenesis in the colon. Cell. Mol. Life Sci. 29, 10.

Su, A. I., Wiltshire, T., Batalov, S., Lapp, H., Ching, K. A., Block, D., Zhang, J., Soden, R., Hayakawa, M., Kreiman, G., et al. (2004). A gene atlas of the mouse and human protein-encoding transcriptomes. Proc. Natl. Acad. Sci. U.S.A 101, 6062-6067.

1. A method of determining or confirming chronic inflammatory intestinal disease or a risk thereof in a subject, the method comprising detecting the presence of keratin 7 (K7) mRNA or protein in a stool sample obtained from said subject.

2. (canceled)

3. (canceled)

4. The method according to claim 1, wherein the said inflammatory intestinal disease is inflammatory bowel disease (IBD) or its subtype.

5. The method according to claim 4, wherein said inflammatory bowel disease is Crohn's disease or ulcerative colitis, wherein the presence of keratin 7 (K7) protein in the stool sample confirms that the stool sample is associated with Crohn's disease or ulcerative colitis.

6. The method according to claim 4, further comprising a step of distinguishing inflammatory bowel disease subtypes from collagenous colitis, microscopic colitis and from irritable bowel syndrome, wherein the presence of keratin 7 (K7) protein in the stool sample confirms that the stool sample is associated with inflammatory bowel disease subtypes.

7. The method according to claim 4, further comprising a step of differentiating microscopic colitis from inflammatory bowel disease, wherein the presence of keratin 7 (K7) protein in the stool sample confirms that the stool sample is associated with inflammatory bowel disease.

8. The method according to claim 1, wherein the presence of keratin 7 (K7) protein in the stool sample is determined by immunohistochemistry or immunofluorescence methods.

9. The method according to claim 8, wherein the presence of keratin 7 (K7) protein in the stool sample is determined by contacting said stool sample with a primary antibody specific to K7 and then the stool sample is visualized by further contacting said stool sample with a labelled secondary antibody binding to the primary antibody and a label-specific reagent.

10. The method according to claim 1, wherein the presence of keratin 7 (K7) protein in the stool sample is determined by preparing an RNA or cDNA sample from said stool sample and detecting the presence and optionally the level of the K7 mRNA or cDNA in said RNA or cDNA sample, respectively, and wherein the presence of the K7 mRNA or cDNA indicates that said stool sample is associated with inflammatory bowel disease.

11. The method according to claim 1, wherein the amount of the K7 protein or K7 mRNA detected in said stool sample is compared to the amount of the K7 protein or K7 mRNA detected in corresponding samples taken from healthy population, and wherein higher amount of the K7 protein or K7 mRNA detected in said stool sample than in the samples of healthy population confirms that the stool sample is associated with inflammatory bowel disease.

12. The method according to claim 11, wherein the amount of the K7 protein or K7 mRNA detected in said stool sample is compared with a cutoff value provided by corresponding assays performed to a number of subjects from healthy population, and wherein a value above the cutoff is an indication that the subject has IBD or a risk for developing IBD.

13. The method according to claim 9, wherein the presence of the combination of the primary and secondary antibody is measured using an enzyme-linked immunosorbent assay.

14. The method according to claim 13, wherein the presence of the K7 protein is determined by a qualitative enzyme-linked immunosorbent assay.

15. The method according to claim 13, wherein the presence of the K7 protein is measured quantitatively.

16. The method according to claim 15, further comprising a step of diluting a stool sample in order to improve optical properties of the sample.

17. The method according to claim 14, further comprising a step of contacting the stool sample with immobilized antibodies specific to the K7 protein to create a treated sample.

18. The method according to claim 17, further comprising a step of contacting said treated sample with enzyme-linked antibodies to create a readable sample.

19. The method according to claim 18, further comprising a step of determining the optical density of said readable sample at suitable wavelength.

20. The method according to claim 19, further comprising a step of generating a purified K7 protein standard curve.

21. The method according to claim 202 further comprising a step of comparing said optical density of said readable sample to said standard curve to determine the concentration of the K7 protein in said stool sample.

22. The method according to claim 1, wherein the presence of keratin 7 (K7) protein in the stool sample is determined by flow cytometry or mass cytometry.

23. The method according to claim 1, wherein the presence of keratin 7 (K7) protein in the stool sample is determined by lateral flow assay or nuclear magnetic resonance (NMR).

24. The method according to claim 1, wherein the method further comprises a step of detecting the presence of a further biomarker selected from the group consisting of: keratin 8 (K8) protein keratin 16 (K16) protein, keratin 17 (K17) protein, keratin 18 (K18) protein, keratin 19 (K19) protein, keratin 20 (K20) protein, keratin 23 (K23) protein, keratin 24 (K24) protein, keratin 80 (K80) protein and calprotectin or a mRNA thereof; in said stool sample.

25-27. (canceled)
